(19) United States
(12) Patent Application Publication
(10) Pub. No.: US 2013/0245739 A1
(43) Pub. Date: Sep. 19, 2013

(54) SELF-ANCHORED STIMULATOR LEAD AND METHOD OF INSERTION

(71) Applicant: Anatoly Arber, Highland Park, IL (US)

(72) Inventor: Anatoly Arber, Highland Park, IL (US)

(21) Appl. No.: 13/419,632

(22) Filed: Mar. 14, 2012

(51) Int. Cl. A61N 1/05

(52) U.S. Cl. 607/117; 607/116

(57) ABSTRACT

An implantable electrical lead comprising an elongate lead body comprising first and second ends and an outer housing defining an interior channel. At least one electrical contact is positioned along the elongate lead body. An anchoring system is disposed at one end of the elongate lead body. The anchoring system is movable between an elongate configuration and a compressed configuration. The compressed configuration has a greater diameter than the elongate configuration. A linkage is provided having a first end attached to the anchoring system, an intermediate portion disposed within the interior channel of the elongate lead body, and a second end disposed outside of the elongate lead body adjacent to the second end of the elongate lead body. Movement of the linkage away from the elongate lead body moves the anchoring system from the elongate configuration to the compressed configuration.

### FIELD OF INVENTION

The present invention is related to lead stimulation therapy. More specifically, the present invention is related to implantable medical electrical leads delivering stimulation therapy and to the prevention of lead migration from the insertion site, such as the spinal column or brain.

### BACKGROUND

Patients with intractable back pain that are unresponsive to conventional therapies such as surgical intervention, injection therapy, physical therapy, or narcotic analgesics can be treated with a lead stimulator, which consists of implantable leads with electrodes connected to a pulse generator. The leads, with electrodes, are inserted via an epidural needle into the epidural space. The leads are placed along the spinal cord in the epidural space such that the pulse generator can send an electrical impulse through the leads and electrodes over the spinal cord to have the effect of blocking the pain sensation in the spinal cord.

During conventional spinal cord therapy, the lead of the stimulator is typically secured by an anchor outside of the spinal canal in the fascia with a suture or staples to prevent migration of the lead. However, a common complication in a high percentage of patients treated with spinal cord stimulation (i.e., over twenty percent) is lead migration. Lead migration is caused by natural movements of the body, which promote sliding of the lead in the epidural space. Lead migration can result in loss of stimulation in the painful area of the spine, and may require lead revision, which can increase surgical costs and the risk of infection with each additional surgery.

A need exists for an improved lead anchoring system to securely anchor the lead in a desired location, such as the epidural space or the brain to reduce lead migration. There also exists a need for a system to securely anchor a lead in a desired location with reduced mechanical parts and minimal invasiveness.

### SUMMARY

An embodiment of the present invention is directed to an implantable electrical lead comprising an elongate lead body comprising first and second ends and an outer housing defining an interior channel. At least one electrical contact is positioned along the elongate lead body. An anchoring system is disposed at one end of the elongate lead body. The anchoring system is movable between an elongate configuration and a compressed configuration. The compressed configuration has a greater diameter than the elongate configuration. A linkage is provided having a first end attached to the anchoring system, an intermediate portion disposed within the interior channel of the elongate lead body, and a second end disposed outside of the elongate lead body adjacent to the second end of the elongate lead body. Movement of the linkage away from the elongate lead body moves the anchoring system from the elongate configuration to the compressed configuration.

A method for inserting an implantable electrical lead in an epidural space of a spinal column comprising providing a lead having a proximal end, a distal end, an outer wall, and a hollow interior. The distal end has a self-anchoring system with a plurality of slits in the outer wall defining a plurality of strips. At least one electrical contact positioned on the outer wall. A linkage is provided having a first portion within the hollow interior of the elongate body and connected to the distal end of the elongate body, and a second portion exterior to the proximal end of the elongate body. The method further comprises positioning the lead in a desired location along the spinal column, and pulling the linkage of the catheter to actuate the self-anchoring system by compressing and radially expanding the plurality of strips.

### BRIEF DESCRIPTION OF THE DRAWINGS

The foregoing summary, as well as the following detailed description of the preferred embodiments of the invention will be better understood when read in conjunction with the appended drawings. For purposes of illustrating the invention, there are shown in the drawings embodiments which are presently preferred. It should be understood, however, that the scope of the invention is not limited to precise arrangements and instrumentalities shown.

In the drawings:

FIG. 1 is a perspective view of a self-anchoring lead according to the present invention with the anchoring system in the elongate configuration.

FIG. 1A is a cross-sectional view of the self-anchoring lead of FIG. 1.

FIG. 2 is a perspective view of the self-anchoring lead of FIG. 1 with the anchoring system in the compressed configuration.

FIG. 3 is a transverse cross-sectional view of the spinal column showing the self-anchoring lead of the present invention inserted in the epidural space in the elongate configuration.

FIG. 4 is a transverse cross-sectional view of the spinal column showing the self-anchoring lead of the present invention inserted in the epidural space in the compressed configuration.

FIG. 5 is a perspective view of an alternative embodiment of a self-anchoring lead without a manual locking system.

FIG. 6 is a perspective view of the self-anchoring lead of the present invention in an alternate compressed configuration.

### DETAILED DESCRIPTION OF THE PREFERRED EMBODIMENTS

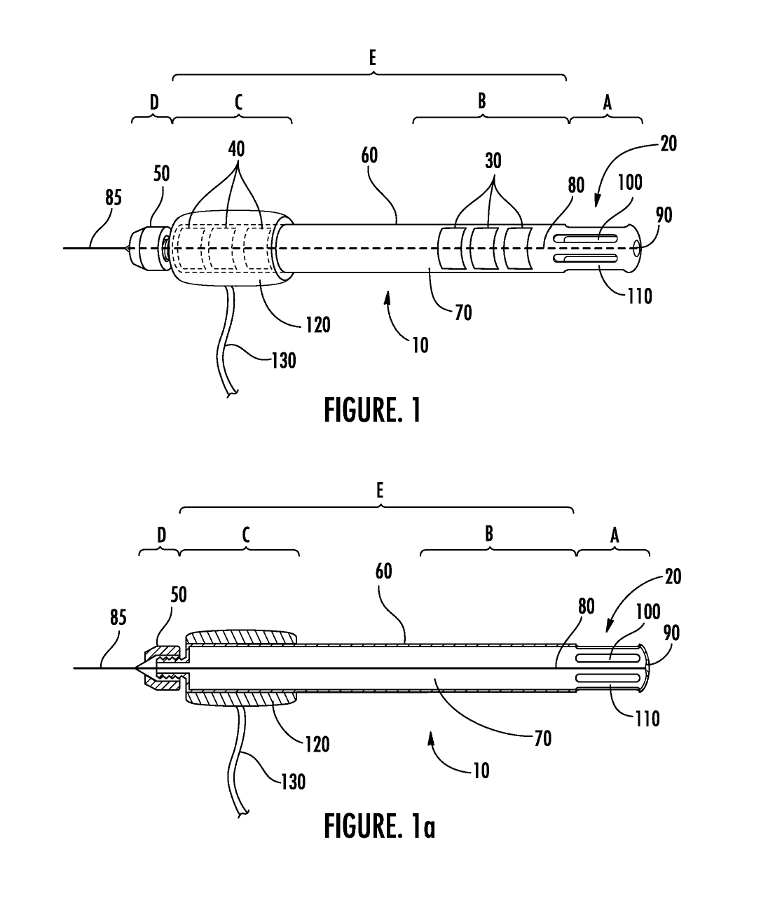

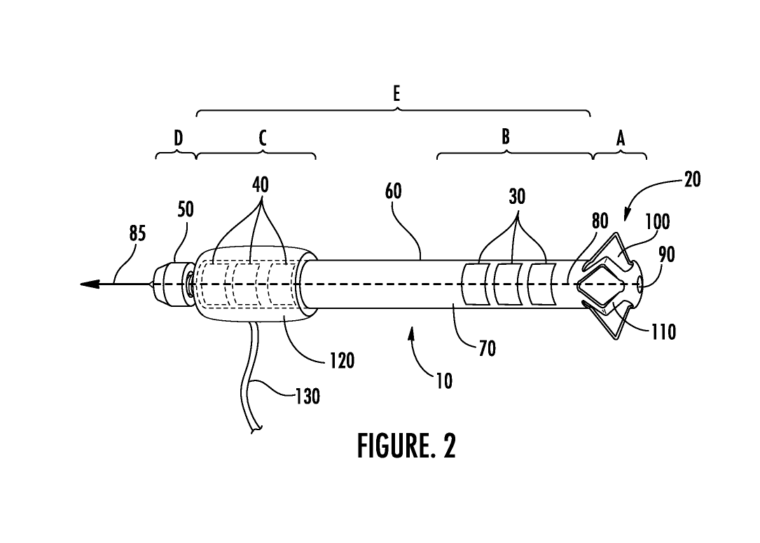

FIGS. 1, 1A, and 2 illustrate a self-anchoring lead or catheter 10 for delivering electrical impulses to a spinal cord according to the present invention. As shown in FIGS. 1 and 1A, the lead 10 includes a distal end (A) in which an anchoring system 20 is located, an elongate body (E), which includes a distal electrode portion (B) with electrical contacts 30 and a proximal electrode portion (C), with electrical contacts 40, and a proximal end (D) in which a locking system 50 can be located. The lead 10 preferably has a housing 60 surrounding an interior channel 70 which houses a linkage 80. Any suitable linkage can be used without departing from the spirit of this invention, including without limitation, a cable or wire. The linkage 80 is secured to the anchoring system at the distal end (A) of the lead, and has a portion 85 that can be exposed outside of the interior channel 70 adjacent to the proximal end (D) of the lead 10. In a preferred embodiment, a metal ball 90 can be attached to the linkage 80 and the anchoring system 20 to secure the linkage 80 to the distal end (A) of the lead 10. However, the linkage 80 can be secured directly to the distal end (A) of the anchoring system 20. The linkage can also be connected to the anchoring system 20 in any other manner understood by one of skill in the art.

Materials and methods of making leads are well known to those skilled in the art. Such techniques include extrusion and coextrusion. The lead 10 of the present invention can be made of any suitable lead material known in the art, including, without limitation, polymeric materials, such as polyamide (nylon), polyurethane, and plastic. Alternatively, the distal end (A) of the lead can be made from a different material than the remainder of the lead 10 so that the anchoring system 20 can be of a material that is softer or more rigid than the remainder of the lead 10. In addition, it is preferred that the lead 10 include a radiopath material to increase the detectability of the lead 10 during surgical implantation of the lead or under X-ray or radio-imaging.

FIGS. 1 and 1A depict the anchoring system in the elongate configuration. The anchoring system 20 is preferably comprised of a distal end (A) of the lead 10 having a plurality of slit or cutouts 100 in the housing 60 such that strips 110 of the housing are provided between the slits 100. The size and number of slits 100 can vary depending on the anatomy of the patient, the intended location for insertion, such as the spinal column or brain, and the intended rigidity/flexibility of the anchoring system 20.

The distal and proximal electrode contacts 30, 40 deliver the electrical current from an electrical source, such as a pulse generator (not shown), to the spinal cord. Proximal electrode contacts 40 can be enveloped by a connector 120 having an extension 130 that leads to the electrical source. Any suitable electrical source can be used without departing from the spirit of this invention, including without limitation, a battery pack that is implanted under the skin or a battery pack that is external to the body.

Those of skill in the art will recognize that the dimensions of the lead 10 can vary based on factors which can include, without limitation, the anatomy of the patient (i.e., size of the epidural space or the brain) and the intended insertion location, such as the spinal column or brain. For example, in spinal cord lead therapy applications, the length of the lead 10 typically ranges from 10 to 100 centimeters, and more typically between 30 and 65 cm. The outer diameter of the lead 10 is typically between 0.5 and 5 millimeters, and more typically 1.0 to 2.5 mm. The linkage 80 is longer than the lead, and is preferably about 1-5 centimeters longer than the lead, and more preferably 2 centimeters longer than the lead.

FIG. 2 illustrates the lead 10 with the anchoring system in the compressed configuration to secure the lead 10 in the desired location, such as the epidural space or brain. Pulling or moving the linkage 80 away from the lead 10 stresses the strips 110 of the anchoring system 20 to compress and radially expand to have an increased diameter as compared to the elongate configuration. When the linkage 80 of the lead 10 is pulled, the strips 110 will radially expand into a shape that is best for the anatomical space in which the lead 10 was inserted. For example, in certain instances, only one half of the self-anchoring system 20 may expand when half of the lead 10 meets resistance such that it cannot expand. The slits 100 in the self-anchoring system 20 can be arranged in a manner to provide the proper shape for the anatomical space in which the lead will be inserted. For example, there could be two slits 100 and two strips 100 wherein the expanded configuration of the anchoring system 20 will have two primary radially expanded portions.

The linkage 80 can be locked in place by engaging locking system 50 to maintain the position of the strips 110. The locking system 50 can comprise any suitable locking system for maintaining the linkage 80 at a desired position. For example, and without limitation, the locking system 50 can consist of an orbital tapering lock or fastener that is secured around a helical ridge of the proximal end (D) of the lead 10.

The length and width of the anchoring system 20 depends on how far the strips 110 are required to radially expand. The width of the anchoring system 20 is also dependent on the anatomy of the patient, the intended insertion location, such as the spinal column or brain, and the size of the space at the desired insertion location. For example, the width of the epidural space generally increases along the spinal column from the cervical vertebrae to the thoracic vertebrae and to the lumbar vertebrae.

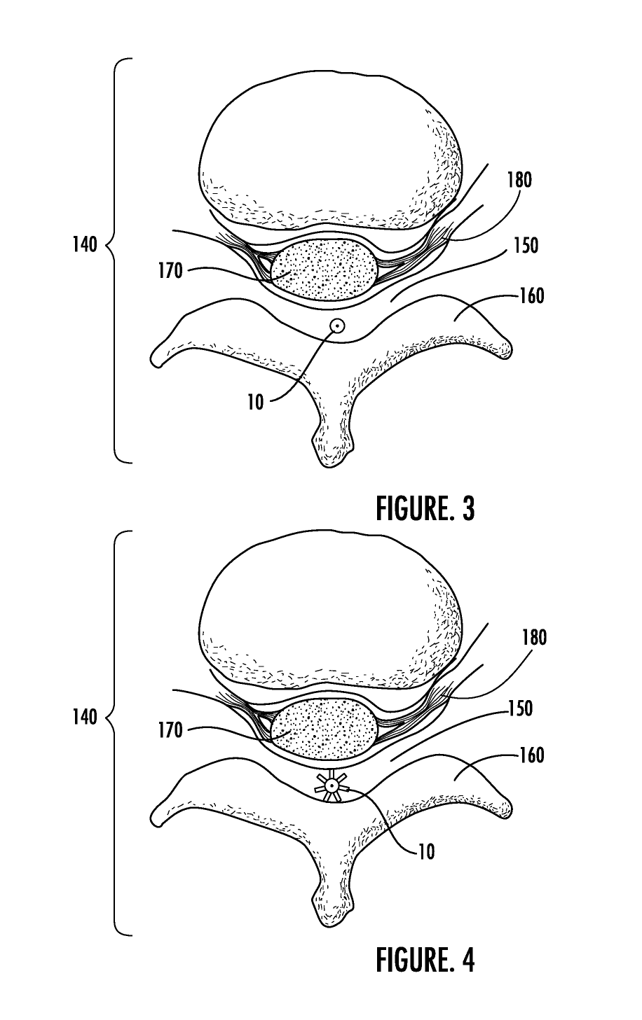

FIG. 3 illustrates a transverse cross-sectional view of a spinal column 140 showing the placement of the lead 10 in its elongate configuration in the epidural space 150 of the spinal column. The spinal column 140 consists of vertebrae 160 and a spinal cord 170 with nerves exiting the spinal cord 180. The epidural space 150 is located between the spinal cord 170 and vertebrae 160. FIG. 4 illustrates a transverse cross-sectional view of the spinal column 140 showing the lead 10 in its expanded configuration in the epidural space 150 of the spinal column 140.

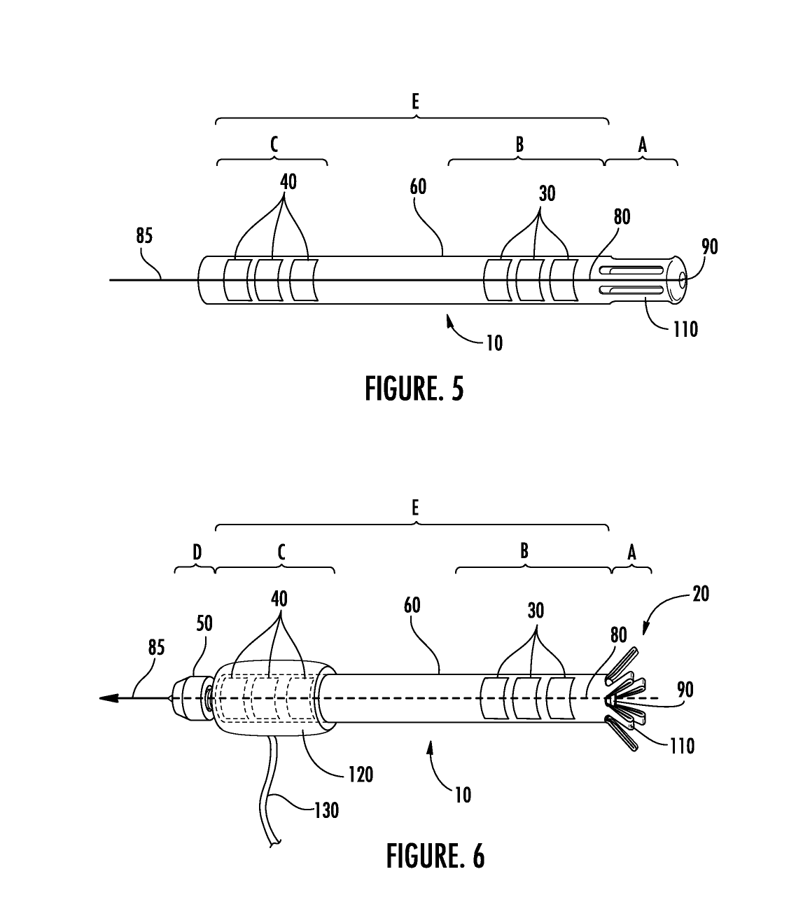

FIG. 5 illustrates an alternate embodiment of the lead 10 without a locking system. In this embodiment, the strips 110 of the anchoring system 20 can be made of a polymeric material that retains its shape upon stress or the exposed portion 85 of the linkage 80 can be wrapped around the lead in order to maintain the anchoring system 20 in the desired position. Alternatively, the self-anchoring system 20 can be made of material that has a residual memory causing it to be biased in an expanded configuration. In order to move the anchoring system 20 to the elongate configuration, the linkage 80 must be pushed toward the distal end of the lead 10.

FIG. 6 illustrates the strips of the anchoring system 20 in an alternate compressed configuration. As shown, the linkage 80 is moved or pulled away from the lead 10 to a greater degree such that the strips 110 fully collapse upon themselves.

The lead 10 according to the present invention may be delivered to the implantation site in the spinal column using methods well-known to those skilled in the art. The lead 10 may be inserted into a patient with an epidural needle. The epidural needle is placed in the epidural space 150 at the appropriate location along the spinal column 140 and the lead 10 is then inserted through the needle into the epidural space 140 at the desired location along a portion of the spinal cord that requires stimulation. During the initial positioning of the lead 10, a trial connector can be connected to a stimulation programmer for trial stimulation to determine the final position of the stimulating lead 10. The trial connector is placed on the proximal part of the lead 10 about the proximal electrode contacts 40. Once the preferred location for the lead is determined, the trial connector 120 is detached from the lead 10, the skin and subcutaneous tissue is cut down until fascia is exposed and the needle is removed.

Once the trial connector is removed, a permanent connector 120 and extension 130 is inserted and connected with the lead 10 by inserting the proximal portion of the lead 10 and the linkage 80 through the opening of the connector 120 and the connector 120 is moved along the lead 10 until the proximal electrode contacts of the lead 10 and the connector 130 are aligned. The attached extension 130 will lead out to an electrical source. Once the connector 120 and extension 130 are in place, the linkage will extend out from the locking system 50. Pulling or moving the linkage 80 away from the lead stresses the strips 110 of the anchoring system 20 to compress and radially expand to have an increased diameter as compared to the elongate configuration. The linkage 80 can be locked in place by engaging the locking system 50.

The lead anchoring system of the present invention reduces lead migration with minimal parts and minimal invasiveness.

While various methods, configurations, and features of the present invention have been described above and shown in the drawings, one of ordinary skill in the art will appreciate from this disclosure that any combination of the above features can be used without departing from the scope of the present invention. It is also recognized by those skilled in the art that changes may be made to the above described methods and embodiments without departing from the broad inventive concept thereof.

1. An implantable electrical lead comprising:
an elongate lead body comprising first and second ends and an outer housing defining an interior channel;
at least one electrical contact positioned along the elongate lead body;
an anchoring system disposed at one end of the elongate lead body, the anchoring system movable between an elongate configuration and a compressed configuration, the compressed configuration having a greater diameter than the elongate configuration;
a linkage having a first end attached to the anchoring system, an intermediate portion disposed within the interior channel of the elongate lead body, and a second end disposed outside of the elongate lead body adjacent to the second end of the elongate lead body;
wherein movement of the linkage away from the elongate lead body moves the anchoring system from the elongate configuration to the compressed configuration.

2. The implantable electrical lead of claim 1, wherein the implantable electrical lead is a spinal cord stimulator lead.

3. The implantable electrical lead of claim 1, further comprising a locking assembly disposed at the second end of the elongate lead body to maintain a portion of the linkage in a fixed position relative to the second end of the elongate lead body to create resistance on the anchoring system.

4. The implantable electrical lead of claim 3, wherein the locking assembly comprises an orbital tapering lock.

5. The implantable electrical lead of claim 1, wherein the linkage comprises a wire or a cable.

6. The implantable electrical lead of claim 1, wherein a metal ball is fixed to the anchoring system and connected to the linkage.

7. The implantable electrical lead of claim 1, wherein the anchoring system is comprised of a polymeric material.

8. The implantable electrical lead of claim 1, wherein the anchoring system comprises an exterior housing defining a hollow interior, the exterior housing comprising a plurality of strips of flexible material positioned between a plurality of slits.

9. The implantable electrical lead of claim 8, wherein in the strips are compressed to fold back upon themselves and radially expand in the collapsed configuration.

10. An implantable electrical stimulator lead comprising:
an elongate body having a proximal end, a distal end, an outer wall and a hollow interior, the distal end having a plurality of slits disposed in the outer wall defining a plurality of strips in the outer wall, the plurality of strips being made of a flexible material;
at least one electrical contact disposed on the outer wall;
an extension connectable to an electrical source; and
a linkage having a first portion within the hollow interior of the elongate body and connected to the distal end of the elongate body, and a second portion exterior to the proximal end of the elongate body.

11. The lead of claim 10 wherein the proximal end of the elongate body includes a lock to maintain the linkage in a fixed position to create resistance on the anchoring system.

12. The lead of claim 11, wherein the lock is a tapering orbital lock.

13. The lead of claim 10, wherein the linkage is a wire.

14. The lead of claim 13 wherein movement of the wire away from the proximal end of the elongate lead body causes the strips of the self-anchoring system to compress and radially expand.

15. The lead of claim 14 wherein the wire is maintained in a fixed position by a lock attached to the elongate body to create resistance on the anchoring system.

16. A method for inserting an implantable electrical lead in an epidural space of a spinal column comprising:
providing a lead having a proximal end, a distal end, an outer wall, and a hollow interior, the distal end having a self-anchoring system with a plurality of slits in the outer wall defining a plurality of strips, at least one electrical contact positioned on the outer wall, and a linkage having a first portion within the hollow interior of the elongate body and connected to the distal end of the elongate body, and a second portion exterior to the proximal end of the elongate body;
positioning the lead in a desired location along the spinal column; and
pulling the linkage of the catheter to actuate the self-anchoring system by compressing and radially expanding the plurality of strips.

17. The method as claimed in claim 16 wherein placing the lead in an epidural needle, and wherein the epidural needle is placed in the epidural space along a segment of the spinal cord that requires stimulation.

18. The method as claimed in claim 16 wherein the lead has a connector connected to an electrical source.

19. The method as claimed in claim 16 wherein the proximal end of the elongate body includes a lock to maintain the linkage in a fixed position.

20. The method as claimed in claim 16 wherein movement of the linkage away from the proximal end of the elongate lead body causes the strips of the self-anchoring system to compress and radially expand.
